(19) United States
(12) Patent Application Publication
(10) Pub. No.: US 2010/0032241 A1
(43) Pub. Date: Feb. 11, 2010

(54) Lubricating Systems

(76) Inventors: Brian Lawrence, Oxfordshire (GB); Christopher Locke, Oxfordshire (GB)

Correspondence Address: INFINEUM USA L.P., P.O. BOX 710, LINDEN, NJ, 07036, US

(21) Appl. No.: 11/722,491

(22) PCT Filed: Dec. 13, 2005
(86) PCT No.: PCT/EP05/13422

§ 371 Date: Oct. 10, 2008

(30) Foreign Application Priority Data
Dec. 24, 2004 (GB) 0428284.4

(51) Int. Cl. F01M 1/02; F02M 25/07; C07D 207/26

(52) U.S. Cl. 184/6.5; 123/568.11; 508/268

(57) ABSTRACT

A lubricant containing a minor amount of a polyalkene-substituted succinimide of Mn 1800 to 2800, as a first base, is circulated through a lubricating system so that the succinimide forms salts with at least a portion of combustion acids therein. The lubricant is then contacted with an immobilised second base, stronger than the first base, that displaces at least a portion of the succinimide from the salts to form retained second base: combustion acid salts and release the succinimide into the lubricant.

This invention relates to lubrication of or for an internal combustion engine, especially a reciprocating piston engine such as a compression-ignited or a spark-ignited engine.

Internal combustion engines are lubricated by circulating lubricating oil (or crankcase lubricant) from an oil sump that is generally or usually situated below the crankshaft of the engine; fuel combustion acids that enter the lubricant must be neutralized to optimise functioning of the engine. It is known to do this by incorporating metal-containing detergent additives in the lubricant. Such detergents are effective in reducing piston deposits but a limit may be reached where further reduction of piston deposits becomes increasingly more difficult to achieve.

U.S. Pat. No. 5,164,101 describes a way of overcoming the above problem. The lubricant includes a weak base to neutralise all or part of the combustion acids and form weak base: combustion acid salts. These salts pass to an immobilised strong base which displaces the weak base and releases it for recirculation in the lubricant. The strong base: combustion acid salts thereby formed are immobilised and prevented from contributing to piston deposits.

U.S. Pat. No. 5,164,101 states that suitable weak bases also include polybutenyl succinimides of polyamines where the polybutenyl group has a number average molecular weight (Mn) from about 900 to about 5000 and that preferably, Mn will range from about 900 to about 1300. In Example 3 thereof, use of “polybutenyl succinimide of polyamine” is described but the molecular weight of the polybutenyl group is not stated.

This invention provides, surprisingly, a significant improvement over the teaching of U.S. Pat. No. 5,154,101 when employing succinimides in which the polybutenyl group has a higher and selected Mn. Moreover, the improvement provided by the present invention is unexpected and contrary to the statement made in US-A1-2004/0050373 that it is expected, in the context of use of polyethyleneamine amide of polybutenyl succinic anhydride, that the method of using strong base in the oil filter will be insufficient and short-lived.

In a first aspect, this invention provides a lubricating system for an internal combustion engine comprising:
(A) a lubricant and means for circulating the lubricant, the lubricant having a phosphorus content, expressed as atoms of phosphorus, of less than 0.1 mass % and an ash content, expressed as sulphated ash, of less than 1.0 mass %, and containing, in a minor amount, a first base comprising a polyalkene-substituted succinimide wherein the polyalkene group has a number-average molecular weight as measured by gel permeation chromatography in the range of 1800 to 2800, the succinimide being capable of neutralizing at least a portion of fuel combustion acids in the lubricant to form, in solution in the oil, a salt or salts of the succinimide and the acids, and
(B) a second base immobilised in the lubricating system capable of displacing at least a portion of the succinimide from the salt or salts to form and retain a salt or salts of the strong base and the acids so that the succinimide thereby released enters the lubricant.

In a second aspect, the invention provides a method of lubricating an internal combustion engine in operation of the engine comprising:
(A) lubricating the engine with a circulating lubricant that
   (A1) has a phosphorus content, expressed as atoms of phosphorus, of less than 0.1 mass %;
   (A2) has an ash content, expressed as sulphated ash, of less than 1.0 mass %; and
   (A3) contains, in a minor amount, a first base comprising a polyalkene-substituted succinimide wherein the polyalkene group has a number-average molecular weight as measured by gel permeation chromatography in the range of 1800 to 2800, such that the succinimide neutralizes at least a portion of fuel combustion acids in the lubricant to form, in solution in the lubricant, a salt or salts of the succinimide and the acids; and
(B) contacting the lubricant from step (A) with an immobilised, second base, stronger than the first base, so that the second base displaces at least a portion of the succinimide from the salt or salts to form and retain a salt or salts of the second base and the acids, whereby the succinimide released enters the lubricant.

This aspect of the invention preferably further comprises, and shows particular advantages in the presence of, the additional step (step (C)) of re-circulating exhaust combustion gases to the engine's intake.

In a third aspect, the invention provides the use, in a lubricating system of or for an internal combustion engine, to improve the control of piston deposits in the engine, of an immobilised second base to displace at least a portion of a first base, weaker than the second base, and comprising a polyalkene-substituted succinimide wherein the polyalkene group has a number-average molecular weight as measured by gel permeation chromatography in the range of 1800 to 2800, from first base: acid salts in circulating lubricant.

In the fourth aspect, the invention provides the use, in a lubricating system of or for an internal combustion engine, of a polyalkene-substituted succinimide wherein the polyalkene group has a number-average molecular weight as measured by gel permeation chromatography in the range of 1800 to 2800, in circulating lubricant to improve the control of piston deposits in the engine, the system including an immobilised second base, stronger than the succinimide, to displace at least a portion of the succinimide from succinimide:acid salts.

In relation to each of the first, second, third and fourth aspects of the invention, the immobilised second base preferably consists essentially of magnesium oxide.

A further advantage of the invention is that at least a portion of acids are eliminated from the circulating lubricant, but in a manner which prevents or limits metallic components from interfering with the operation of any exhaust particulate filter or other emissions control device that may be used.

A yet further advantage of the invention is that the combination of polyalkene-substituted succinimide wherein the polyalkene group has a number-average molecular weight in the range of 1800 to 2800 in circulating lubricant and immobilised base as defined herein (and preferably consisting essentially of magnesium oxide) permits the operation of an internal combustion engine (and particularly an engine wherein, in operation, exhaust combustion gases are re-circulated or reconsumed, preferably with associated cooling, to the engine's air intake) with a lubricating oil having the low phosphorus and sulphur contents defined by A1 and A2 above. Reconsumption of exhaust gases may, for example, be affected by variable valve timing as in so-called “Internal” exhaust gas recirculation (Internal EGR).

In this specification, the following words and expressions, if and when used, shall have the meanings ascribed below:
“active ingredient” or “(a.i.)” refers to additive material that is not diluent or solvent;
“comprising” or any cognate word specifies the presence of stated features, steps, or integers or components, but does not preclude the presence or addition of one or more other features, steps, integers, components or groups thereof; the expressions “consists of” or “consists essentially of” or cognates may be embraced within “comprises” or cognates, wherein “consists essentially of” permits inclusion of substances not materially affecting the characteristics of the composition to which it applies;
“major amount” means in excess of 50 mass % of a composition;
“minor amount” means less than 50 mass % of a composition;
“TBN” means total base number as measured by ASTM D4739;
“TAN” means total acid number as measured by ASTM D664.

Furthermore, in this specification:
“phosphorous content” is as measured by ASTM D5185;
“sulphated ash content” is as measured by ASTM D874; and
“sulphated content” is as measured by ASTM D2622.

Also, it will be understood that various components used, essential as well as optimal and customary, may react under conditions of formulation, storage or use and that the invention also provides the product obtainable or obtained as a result of any such reaction.

Further, it is understood that any upper and lower quantity, range and ratio limits set forth herein may be independently combined.

The features of the invention relating, where appropriate, to each and all aspects of the invention, will now be described in more detail as follows:

### Lubricant

The lubricant contains a major proportion of an oil of lubricating viscosity (sometimes referred to as “base stock” or “base oil”) as the primary liquid constituent of the lubricant into which additives and possibly other oils are blended.

A base oil may be selected from natural (vegetable, animal or mineral) and synthetic lubricating oils and mixtures thereof. It may range in viscosity from light distillate mineral oils to heavy lubricating oils such as gas engine oil, mineral lubricating oil, motor vehicle oil and heavy duty diesel oil. Generally the viscosity of the oil ranges from 2 to 30, especially 5 to 20, mm²s⁻¹ at 100° C.

Natural oils include animal and vegetable oils (e.g. castor and lard oil), liquid petroleum oils and hydrorefined, solvent-treated mineral lubricating oils of the paraffinic, naphthenic and mixed paraffinic-naphthenic types. Oils of lubricating viscosity derived from coal or shale are also useful base oils.

Synthetic lubricating oils include hydrocarbon oils such as polymerized and interpolymerized olefins (e.g. polybutylenes, polypropylenes, propylene-isobutylene copolymers, chlorinated polybutylenes, poly(1-hexenes), poly(1-octenes), poly(1-decenes)); alkylbenzenes (e.g. dodecylbenzenes, tetradecylbenzenes, dinonylbenzenes, di(2-ethylhexyl)benzenes); polyphenols (e.g. biphenyls, terphenyls, alkylated polyphenols); and alkylated diphenyl ethers and alkylated diphenyl sulfides and derivatives, analogues and homologues thereof.

Another suitable class of synthetic lubricating oils comprises the esters of dicarboxylic acids (e.g. phthalic acid, succinic acid, alkyl succinic acids and alkenyl succinic acids, maleic acid, azelaic acid, suberic acid, sebasic acid, fumaric acid, adipic acid, linoleic acid dimer, malonic acid, alkylmalonic acids, alkenyl malonic acids) with a variety of alcohols (e.g. butyl alcohol, hexyl alcohol, dodecyl alcohol, 2-ethylhexyl alcohol, ethylene glycol, diethylene glycol monoether, propylene glycol). Specific examples of these esters include dibutyl adipate, di(2-ethylhexyl) sebacate, di-n-hexyl fumarate, dioctyl sebacate, diisooctyl azelate, diisodecyl azelate, dioctyl phthalate, didecyl phthalate, dieicosyl sebacate, the 2-ethylhexyl diester of linoleic acid dimer, and the complex ester formed by reacting one mole of sebacic acid with two moles of tetraethylene glycol and two moles of 2-ethylhexanoic acid.

Esters useful as synthetic oils also include those made from C₅ to C₁₂ monocarboxylic acids and polyols, and polyol ethers such as neopentyl glycol, trimethylolpropane, pentaerythritol, dipentaerythritol and tripentaerytritol.

Unrefined, refined and re-refined oils can be used in the lubricants of the present invention. Unrefined oils are those obtained directly from a natural or synthetic source without further purification treatment. For example, a shale oil obtained directly from retorting operations, a petroleum oil obtained directly from distillation or ester oil obtained directly from an esterification process and used without further treatment would be unrefined oil. Refined oils are similar to the unrefined oils except they have been further treated in one or more purification steps to improve one or more properties. Many such purification techniques, such as distillation, solvent extraction, acid or base extraction, filtration and percolation are known to those skilled in the art. Re-refined oils are obtained by processes similar to those used to obtain refined oils applied to refined oils which have been already used in service. Such re-refined oils are also known as reclaimed or reprocessed oils and often are additionally processed by techniques for approval of spent additive and oil breakdown products.

Other examples of base oil are gas-to-liquid (“GTL”) base oils, i.e. the base oil may be an oil derived from Fischer-Tropsch-synthesised hydrocarbons made from synthesis gas containing hydrogen and carbon monoxide using a Fischer-Tropsch catalyst. These hydrocarbons typically require further processing in order to be useful as a base oil. For example, they may, by methods known in the art, be hydroisomerized; hydrocracked and hydroisomerized; dewaxed; or hydroisomerized and dewaxed.

Base oil may be categorised in Groups 1 to V according to the API EOLCS 1509 definition.

The oil of lubricating viscosity is provided in a major amount, in combination with a minor amount of at least one additive and, if necessary, one or more co-additives such as described hereinafter, constituting the lubricant. This preparation may be accomplished by adding the additive directly to the oil or by adding it in the form of a concentrate thereof to disperse or dissolve the additive. Additives may be added to the oil by any method known to those skilled in the art, either prior to, contemporaneously with, or subsequent to, addition of other additives.

The terms “oil-soluble” or “dispersible”, or cognate terms, used herein do not necessarily indicate that the compounds or additives are soluble, dissolvable, miscible, or are capable or being suspended in the oil in all proportions. They do mean, however, that they are, for instance, soluble or stably dispersible in oil to an extent sufficient to exert their intended effect in the environment in which the oil is employed. Moreover, the additional incorporation of other additives may also permit incorporation of higher levels of a particular additive, if desired.

The lubricant may have a sulphur content of less than 0.4 mass %.

### First Base

The lubricant, as stated, contains, in a minor amount, a first base comprising a polyalkene-substituted succinimide wherein the polyalkene group has a number-average molecular weight in the range of 1,800 to 2800. The number-average molecular weight is measured, as stated above, by gel permeation chromatography (GPC). The polyalkene group may comprise a major molar amount (i.e. greater than 50 mole %) of a C₂ to C₁₈ alkene, e.g. ethene, propene, butene, isobutene, pentene, octane-1 and styrene. Preferably, the alkene is a C₂ to C₅ alkene; more preferably it is butene or isobutene, such as may be prepared by polymerisation of a C₄ refinery stream. Preferably, the number average molecular weight of the polyalkene group is in the range of 2,000 to 2,500, such as 2,200 to 2,400.

The succinimides may be prepared by reacting a polyalkene-substituted succinic acid, or derivative, with a nitrogen-containing compound such as a polyalkene polyamine, for example having the general formula

H₂N([C₂H₄]ₙNH)ₘH

wherein:
m is an integer from 2 to 20, and
n is an integer from 1 to 6.

The succinimides may be post-treated, for example borated, by methods that may be known in the art.

The first base will normally be added to the lubricant during its formulation or manufacture. It must be strong enough to neutralize the combustion acids (i.e. form a salt). Suitable first bases will typically have a pKa from 4 to 12 and may be termed “weak bases”.

The first base should be sufficiently soluble for the salt or salts formed to remain soluble in the lubricant and not to precipitate.

The amount of first base in the lubricant will vary depending upon the amount of combustion acids present, the degree of neutralization desired, and the specific applications of the lubricant. In general, the amount need only be that which is effective or sufficient to neutralize at least a portion of the combustion acids. Typically, the amount will range, as active ingredient, from 0.01 to 3 wt % or more such as up to 4 wt %, preferably from 0.1 to 1.0 wt %.

In addition to the first base and as indicated above, other additives known in the art may be added to the lubricating base oil to form a fully-formulated low ash lubricant. Such lubricating oil additives include other dispersants, antiwear agents, antioxidants, corrosion inhibitors, detergents, pour point depressants, extreme pressure additives, viscosity index improvers and friction modifiers.

### Second Base

Following neutralization of the combustion acids, the neutral salts thereby formed are passed or circulated from the piston ring zone with the lubricant and contacted with the second base. By second base is meant a base that will displace the first base from the neutral salts and return the first base to the lubricant for recirculation to the piston ring zone where the weak base is reused to neutralize combustion acids. Examples of suitable second bases include, but are not limited to, barium oxide, calcium carbonate, calcium oxide, calcium hydroxide, magnesium carbonate, magnesium hydroxide, magnesium oxide, sodium aluminate, sodium carbonate, sodium hydroxide, zinc oxide, or their mixtures; magnesium oxide is particularly preferred. In many cases the second bases may be termed “strong bases”.

The second base may be adhered to or incorporated (e.g. impregnated) on or with a substrate immobilized in the lubricating system of the engine. The substrate can be located on the engine block or near the sump. Preferably the substrate, if used, will be part of the filter system for filtering lubricant, although it could be separate therefrom. Preferred substrates include paper, fabric, felt, glass, plastic, microglass and both woven and non-woven polymeric fibre. Other useful substrates include, but are not limited to, alumina, activated clay, cellulose, cement binder, silica-alumina, and activated carbon. The substrate may be inert or not inert.

The second base may be incorporated into or adhered onto the substrate by methods known to those skilled in the art. For example, if the substrate is alumina, the second base can be deposited by using the following technique. A highly porous alumina is selected. The porosity of the alumina is determined by weighing dried alumina and then immersing it in water. The alumina is removed from the water and the surface water removed by blowing with dry air. The alumina is then reweighed and compared with the dry alumina weight. The difference in weight is expressed as grams of water per gram of dry alumina. A saturated solution of calcium oxide in water is prepared. This solution is then added to the dry alumina in an amount equal to the difference between the weight of the wet and dry alumina. The water is removed from the alumina with heat leaving calcium oxide deposited on the alumina as the product. This preparation can be carried out under ambient conditions, except that the water removal step is performed at about 100° C.

The amount of second base required will vary with the amount of first base in the lubricant and the amount of combustion acids formed during engine operation. However, since the second base is not being continuously regenerated for reuse (unlike the first base), the amount of second base must be at least equal to (and preferably be a multiple of) the equivalent weight of the first base in the lubricant. Therefore, the amount of second base should be from 1 to 15 times, preferably from about 1 to 5 times, the equivalent weight of the first base in the lubricant.

Once the first base has been displaced from the soluble neutral salts, the second base: combustion acid salts thus formed will be immobilized as deposits with the second base, for an example, on the substrate, if used. Thus, deposits which would normally be formed in the piston ring zone are not formed until the soluble salts contact the second base. Preferably, the second base will be located such that it can be easily removed from the lubricating system, e.g. by including it as part of the oil filter system.

### EXAMPLES

The invention will now be particularly described in the following examples which are not intended to limit the scope of the claims hereof.

In the examples, reference will be made to the accompanying drawings, in which

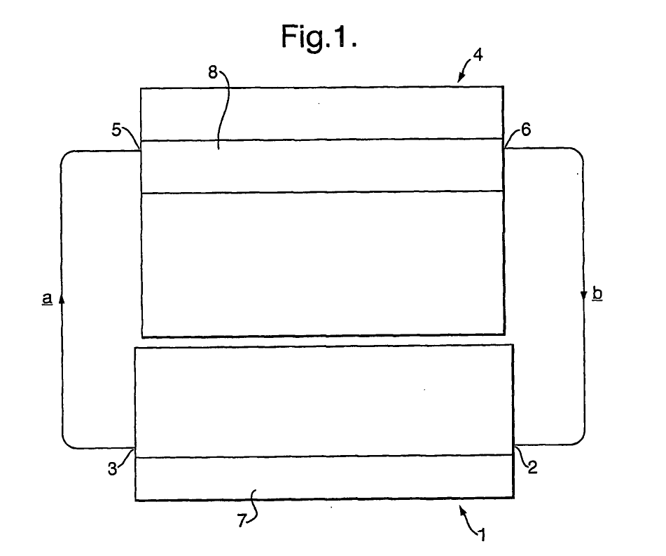

FIG. 1 is a schematic diagram depicting a lubricating system for an internal combustion engine.

Referring to FIG. 1, the system has an internal combustion engine crankcase and oil sump 1 and has a filter 4. The crankcase and sump 1 have an inlet 2 and an outlet 3 for lubricant; lubricant within the crankcase and sump 1 is generally indicated by the numeral 7. The filter 4 has an inlet 5 for lubricant and an outlet 6 for lubricant; located within the filter 4 and immobilised therein is a strong base carried on a substrate 8. Outlet 3 and inlet 5 are interconnected, and outlet 6 and inlet 2 are interconnected to provide a continuous path for lubricant to be circulated through the system in the direction indicated by arrows a and b.

In operation of the system shown in FIG. 1, lubricant, containing a weak base, enters the crankcase and sump 1 in the direction indicated by arrow b at the inlet 2 in order to lubricate the engine. The weak base reacts with at least a portion of combustion acids in the crankcase and sump 1 to form weak base: combustion acid salts, thereby depleting the lubricant in weak base.

Lubricant, depleted in weak base, leaves the crankcase and sump 1 at outlet 3 and enters inlet 5 of filter 4 in the direction shown by arrow a. In the filter 4, the strong base on the substrate 8 displaces at least a portion of the weak base from the salts to form strong base: combustion acid salts which are immobilised on the substrate 8. The lubricant, thus replenished in weak base, leaves the filter at outlet 6 and re-enters the crankcase and sump 1 at inlet 2 in the direction shown by arrow b. At least a portion of the combustion acids are thereby neutralised in the filter 4.

### Preparation of Lubricants

Two fully-formulated 5W30 lubricating oil compositions (or lubricants) were blended by methods know in the art. The two lubricants contained identical components except that Lubricant 1 contained, as the first base, a polybutene-succinimide dispersant wherein the polybutene had an Mn of 2225 and Lubricant A (a reference lubricant) contained a polybutene-succinimide dispersant wherein the polybutene had an Mn of 950. Neither lubricant contained any other source of amine nitrogen and both lubricants contained comparable mass %'s of amine nitrogen (0.051 for Lubricant 1 and 0.054 for Lubricant A) to ensure comparability of dispersing power. Also, the lubricants had comparable TBN's (1.81 for Lubricant 1 and 1.86 for Lubricant A), comparable KV100 (10.17 mm²s⁻¹ for Lubricant 1 and 10.21 mm²s⁻¹ for Lubricant A), and 0.2% sulphated ash.

Each lubricant had a synthetic polyalpha-olefin (PAO) basestock and contained standard commercially-available lubricant components such as one or more metal detergents, anti-wear agents, friction modifiers, none-aminic anti-oxidants, anti-foams and viscosity improvers.

### Testing of Lubricants

The two lubricants were tested in the VW (RTM) turbo-charged direct injection (IDi) engine (1.9 L) test employing the lubricating system shown and described with reference to the accompanying FIG. 1 and wherein, as the second base, the strong base on the substrate, constituting the filter, was magnesium oxide constituting about 25 to 28% by mass of the filter. The selection of the two lubricants was designed to enable the effect attributable to the difference in the molecular weights of the polybutene moieties in the weak base dispersants to be identified and compared.

Results obtained, (measurement of TAN and TBN as a function of time) are set out in the table below.

| | TAN | | TBN | |
|---|---|---|---|---|
| Time (Hours) | Lubricant 1 | Lubricant A | Lubricant 1 | Lubricant A |
| 0 | — | 1.25 | 1.81 | 1.86 |
| 9 | 1.31 | 1.36 | 1.18 | 1.08 |
| 18 | 1.30 | 1.32 | 1.15 | 1.00 |
| 27 | 1.47 | 1.71 | 1.04 | 1.14 |
| 36 | 1.55 | 1.7 | 1.06 | 1.12 |
| 45 | 1.57 | 2.42 | 1.03 | 1.14 |
| 54 | 2.32 | 2.96 | 1.05 | 1.12 |

In the lubricating system employed, Lubricant 1 (of the invention) showed a significantly greater ability than Lubricant A (reference) to control TAN.

1. A lubricating system for an internal combustion engine comprising:
(A) a lubricant and means for circulating the lubricant, the lubricant having a phosphorus content, expressed as atoms of phosphorus, of less than 0.1 mass % and an ash content, expressed as sulphated ash of less than 1.0 mass %, and containing, in a minor amount, a first base comprising a polyalkene-substituted succinimide wherein the polyalkene group has a number-average molecular weight as measured by gel permeation chromatography in the range of 1800 to 2800, the succinimide being capable of neutralizing at least a portion of fuel combustion acids in the lubricant to form, in solution in the oil, a salt or salts of the succinimide and the acids, and
(B) a second base immobilised in the lubricating system capable of displacing at least a portion of the succinimide from the salt or salts to form and retain a salt or salts of the strong base and the acids so that the succinimide thereby released enters the lubricant.

2. A system as claimed in claim 1 wherein the first base comprises a polyisobutene-substituted succinimide.

3. A system as claimed in claim 1 wherein the second base is magnesium oxide.

4. A system as claimed in claim 1 wherein the second base is carried on a substrate.

5. A system as claimed in claim 1 further comprising a device for re-circulating exhaust combustion gases, arising from operation of the engine, to the engine's intake.

6. A system as claimed in claim 1 wherein the engine is a compression-ignited engine.

7. A system as claimed in claim 4 wherein the substrate is part of a oil filter system.

8. A method of lubricating an internal combustion engine in operation of the engine comprising:
(A) lubricating the engine with a circulating lubricant that
(A1) has a phosphorus content, expressed as atoms of phosphorus, of less than 0.1 mass %;
(A2) has an ash content, expressed as sulphated ash of less than 1.0 mass %; and
(A3) contains, in a minor amount, a first base comprising a polyalkene-substituted succinimide wherein the polyalkene group has a number-average molecular weight as measured by gel permeation chromatography in the range of 1800 to 2800, such that the succinimide neutralizes at least a portion of fuel combustion acids in the lubricant to form, in solution in the lubricant, a salt or salts of the succinimide and the acids; and
(B) contacting the lubricant from step (A) with an immobilised, second base, stronger than the first base, so that the second base displaces at least a portion of the succinimide from the salt or salts to form and retain a salt or salts of the second base and the acids, whereby the succinimide released enters the lubricant.

9. A method as claimed in claim 8 comprising the further step of (C) recirculating exhaust combustion gases to the engine's intake.

10. A method as claimed in claim 8 comprising the further step of (C1) reconsuming exhaust combustion gases.

11. (canceled)

12. (canceled)
